(19) United States
(12) Patent Application Publication
(10) Pub. No.: US 2013/0085389 A1
(43) Pub. Date: Apr. 4, 2013

(54) METHOD AND APPARATUS FOR ANORECTAL EXAMINATION

(71) Applicants: Charles Bih Shiou Tsang, Singpore (SG); Feng Liu, Singapore (SG); Di Xiao, Brisbane (AU); Shiow Yunn Lim, Singapore (SG)

(72) Inventors: Charles Bih Shiou Tsang, Singpore (SG); Wan Sing Ng, Singapore (SG); Feng Liu, Singapore (SG); Di Xiao, Brisbane (AU)

(21) Appl. No.: 12/530,636

(22) PCT Filed: Mar. 17, 2008
(86) PCT No.: PCT/SG2008/000082

§ 371 Date: Jun. 18, 2012

Related U.S. Application Data
(60) Provisional application No. 60/895,275, filed on Mar. 16, 2007.

(51) Int. Cl. A61B 8/08

(52) U.S. Cl. CPC A61B 8/48
USPC 600/442

(57) ABSTRACT

An anorectal probe system comprising; an anorectal probe assembly having an insertion end for insertion into a patient's rectum, said probe assembly including a transducer at or adjacent to the insertion end for collecting ultrasound data; a mounting in rotational engagement with said probe assembly at a point distal from the insertion end; said mounting pivotally coupled to the probe assembly at a point intermediate the insertion end and rotational engagement point; wherein on application of a moment about said pivotal coupling, said mounting and probe assembly are arranged to permit selective pivotal movement of the probe assembly about the coupling.

### FIELD OF INVENTION

The invention relates to anorectal examination, and methods and apparatus to achieve this. In particular the invention relates to the use of ultrasound methods to acquire a series of two dimensional images and create a 3-dimensional image so as to more readily identify tumors leading to rectal cancer.

### BACKGROUND OF THE INVENTION

Cancer management is most effective when the cancer is diagnosed early and hence treated early. This is especially true of rectal cancer as it requires surgery in nearly all cases for a complete cure, although radiation and chemotherapy are sometimes used in addition to surgery. Hence, an Endoscopic Ultrasonography (EUS) method is invaluable as a diagnostic tool in rectal cancer in obtaining valuable information about the tumor.

Traditionally, the EUS procedure involves inserting an ultrasound probe manually by the surgeon through the patient's anus in order to scan the rectum. During the procedure, the surgeon adjusts the probe to maintain the transducer at the centroid of a 2D image slice and records down the desired ultrasound image by using a footpad. If the probe is not positioned at the center of the rectum, the ultrasound image produced will appear fuzzy in certain sections of the image. Therefore, it is important for a surgeon to be able to position and maneuver the probe to the center of the rectal canal in order to obtain a clear radial image.

EUS procedure requires skillful surgeons to place and maneuver the ultrasound probe around the right location to extract the most accurate information for clinical diagnosis. Large amount of information is required to make the right diagnosis rendering a lot of time wasted and causing a lot of discomfort to the patient.

The basic concept of 3D anorectal ultrasound scan is to acquire a series of 2D image slices with tracking the location of each 2D image slice. To date, linear pull back mover (or internal mover) and Magnetically-Tracked Freedhand (MTF) are two common approaches for conducting 3D ultrasound scan. A linear pull back mover is a computer-controlled, motor-driven probe positioner used to move the probe mounted on it backwards to produce a series of parallel images. Similarly, an internal mover (inside the sheath of the probe) drives the transducer inside the probe directly for a series of parallel image acquisition. The MTF approach still needs the skills and experience from an operator to control the position of the transducer for good image quality as mentioned in preceding paragraphs. The shortcoming of the external or internal pull back movers is that the approach cannot keep the transducer at the center of a rectal canal, thereby causing unsatisfactory image quality. It is possible to use untracked freehand probe, where the probe is moved by the operator. It is currently favored by the surgeon. Unfortunately, this does not provide accurate position information and is therefore without benefit for further 3D reconstruction and display.

In many case, a sigmoidoscope will be inserted in advance of inserting the ultrasound probe. The main purpose of the sigmoidoscope is to prevent relative motion between the ultrasound probe and rectal wall to reduce possible discomfort or tearing of the tumor. Hence, the sigmoidoscope is useful during an anorectal ultrasound scanning.

The present of the sigmoidoscope creates a problem in that it needs an additional hand to hold it and the ultrasound probe together, in order to move them in tandem. That means the surgeon will not be able to perform the scanning on his own. The surgeon will need an assistant to hold the sigmoidoscope and the ultrasound probe together so that he can operate the ultrasound machine and adjusts and withdraw the ultrasound probe to acquire desired quality images.

A further problem arises when the surgeon injects water through a water stand to inflate the balloon inside of patient rectum. This may cause water leakage at a location between the water stand and the balloon. The reason is the seals are often not strong enough to prevent the water leakage. Further, there is a tendency for the balloon to inflate at a location between the water stand the sigmoidoscope when injecting water into balloon to inflate the balloon inside of rectum. The reason is the pressure inside the rectum is too high causing the water flow back so that the balloon inside the rectum will lose the contact with rectum wall.

### SUMMARY OF INVENTION

In a first aspect the invention provides an anorectal probe system comprising; an anorectal probe assembly having an insertion end for insertion into a patient's rectum, said probe assembly including a transducer at or adjacent to the insertion end for collecting ultrasound data; a mounting in rotational engagement with said probe assembly at a point distal from the insertion end; said mounting pivotally coupled to the probe assembly at a point intermediate the insertion end and rotational engagement point; wherein on application of a moment about said pivotal coupling, said mounting and probe assembly are arranged to permit selective pivotal movement of the probe assembly about the coupling.

In a second aspect the invention provides an anorectal probe assembly comprising an anorectal probe; an inflatable membrane positioned over said probe; a water insertion system for inflating the membrane with water; a sigmoidoscope having a central bore for receiving the probe and inflatable membrane and a adaptor selectively engageable with said sigmoidoscope and said probe so as to fix said sigmoidoscope and probe and seal said membrane.

In a third aspect the invention provides a method for centralizing an anorectal probe within a patient's rectum, said probe having an insertion end with an ultrasound transducer at or adjacent to the insertion end for collecting ultrasound data, the method comprising the steps of: positioning the insertion end within the patient's rectum; acquiring an ultrasound image from said transducer locating a centroid of said transducer from the image; locating a centroid of the rectal void from said image; calculating the difference in position of said centroids and instructing a controller to move said probe such that the centroids coincide.

In a fourth aspect the invention provides a method of creating a 3D ultrasound image of a patient's rectum including the steps of: inserting an anorectal probe assembly in a patient's rectum; controlling movement of said probe assembly using a control system in communication with a drive system; positioning said probe such that a centroid of a transducer within said probe coincides with a centroid of a rectal void; acquiring a two dimensional ultrasound image at a first position within said patient's rectum; controlling said probe so as to withdraw the transducer to a next position, repeating said positioning, acquiring and withdrawing steps a predetermined number of iterations and combining said 2 dimensional images so as to create a 3 dimensional image is created.

An automated anorectal ultrasound image acquisition system according to one embodiment of the present invention may include a PC based motion controlled system with a drive system and software control system that facilitates automated capturing of 2D ultrasound image slices for further 3D volume reconstruction and image feature extraction.

In a further embodiment, the motion of the drive system for positioning the probe (or transducer) may be image guided to accurately obtain each slice of ultrasound scan according to the natural anatomy of the rectum.

A system according to a further embodiment may capture a series of consecutive 2D ultrasound image scans for the anorectal tract by programmable position control of the ultrasound transducer.

After the probe is inserted into the patient's rectum by the surgeon, it will be attached to the probe control platform using the acquired ultrasound image, may obtain the centroid of the rectal tract and the probe position and guide the probe control platform to adjust the transducer to the centroid of the rectum. The system may record the desired quality image and withdraw the probe to the next interval until the examination is complete. From the captured 2-dimension data, a 3-dimension model may be re-constructed.

A system according to various embodiments may collectively or separately;
(i) Be able to extract the 2D boundary shape and layer structure in the wall of the rectum automatically;
(ii) Be able to reconstruct the 3D view of the rectum base on the 2D slices whose relative position and orientation are known precisely, hence it may provide an intuitive and detailed anatomical view of the whole rectum or rectal wall;
(iii) Be able to display the 3D image in a “peel off” fashion allowing tumor penetration to be shown with respect to the mucosa;
(iv) Be able to extract the tumor boundary to show the proliferation of the tumor;
(v) Be able to extract external anal sphincter and internal anal sphincter as well as the fistula tract.
(vi) Be able to display the 3D structure of the boundary of rectal wall, the tumor and the anal sphincter in a “peel off” fashion.
(vii) Be able to obtain the centroid of the rectal tract automatically during the withdrawal interval of the procedure allowing consistent contract. Moreover, In a further embodiment, the invention may include software including an image acquisition module.

The image acquisition module may guide the probe control platform and perform the image acquisition process. It may further assist in keeping the probe at the centroid of the rectal balloon area for each acquired image. The difference of these two centroids may be used to guide the x-, y- and z-axis motors' motion in the probe control platform by a designed motion control system.

### BRIEF DESCRIPTION OF DRAWINGS

It will be convenient to further describe the present invention with respect to the accompanying drawings that illustrate possible arrangements of the invention. Other arrangements of the invention are possible, and consequently the particularity of the accompanying drawings is not to be understood as superseding the generality of the preceding description of the invention.

FIG. 1 is an isometric view of an anorectal examination device mounted to a movable platform according to one embodiment of the present invention;

FIG. 2 is an isometric view of the anorectal examination device of FIG. 1;

FIGS. 3A and 3B are various views of an anorectal probe assembly according to an embodiment of the present invention;

FIGS. 4A, B and C are various views of an anorectal examination using a device according to a further embodiment of the present invention;

FIGS. 5A and 5B are ultrasound images of a patient rectum indicator rectal void and probe centroids;

FIG. 6 is a flow chart and screen dump of an anorectal examination;

FIG. 7 is a schematic view of modules for a method according to one embodiment of the present invention;

FIG. 8 is a screen dump of an ultrasound image taken by a device according to the present invention;

FIG. 9 is an elevation view of an anorectal examination assembly according to the prior art;

FIG. 10 is an exploded view of an anorectal examination assembly according to one embodiment of the present invention;

FIGS. 11A and 11B are isometric views of an adaptor according to a further embodiment of the present invention;

FIGS. 12A and 12B are isometric views of the adaptor of FIGS. 11A and 11B mounted to a water stand, and;

FIGS. 13A and 13B are various views of the adaptor of FIGS. 11A and 11B mounted to a sigmoidoscope.

### DESCRIPTION OF PREFERRED EMBODIMENT

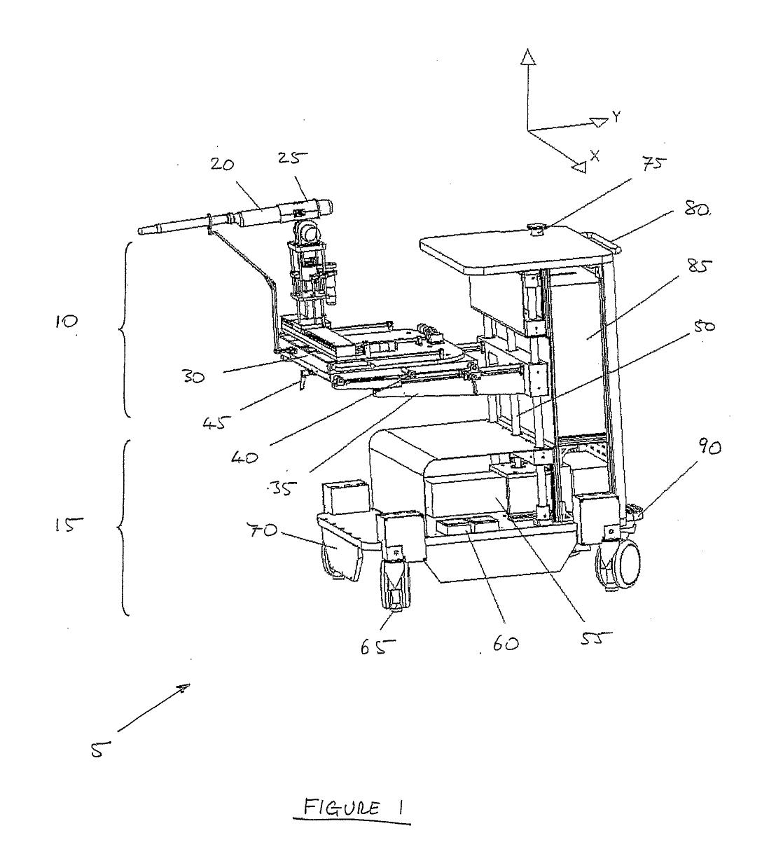

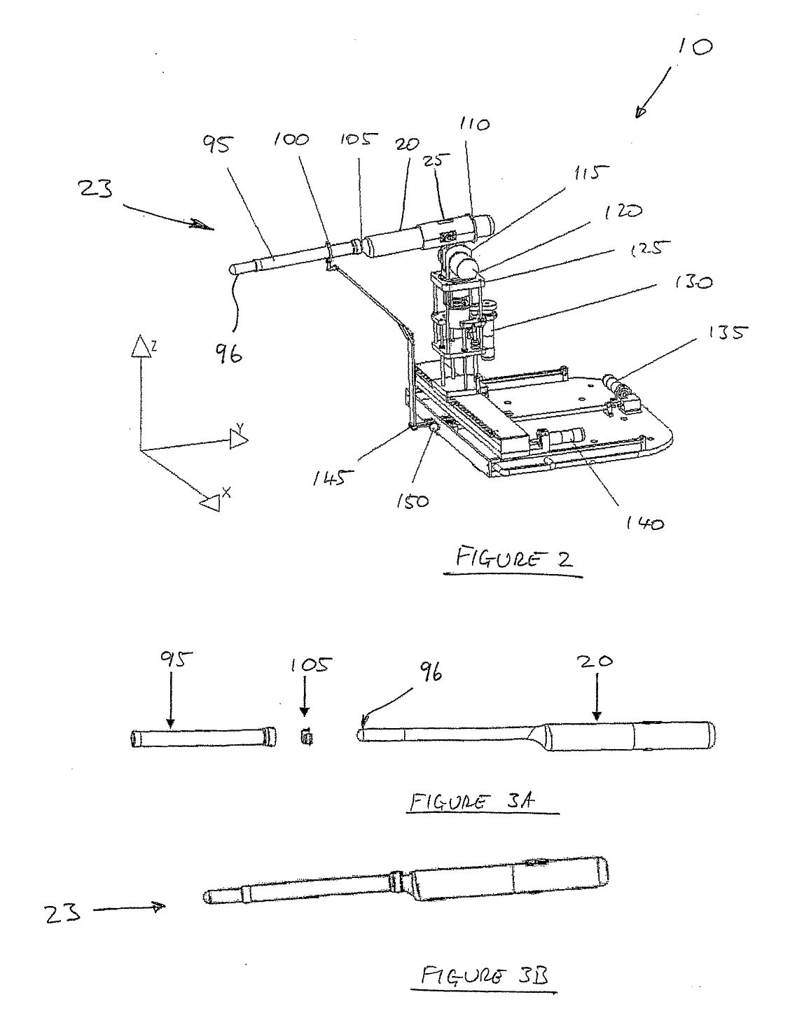

FIGS. 1 and 2 show general arrangements of the present invention according one embodiment. Shown is an anorectal examination device 5 comprising an anorectal examination system 10 mounted to a trolley 15. The system 10 includes an anorectal probe assembly 23 mounted to a mechanical arrangement, in this case, a mounting designed to apply controlled forces, and consequently movement of the probe assembly 23.

The mounting comprises a platform 35, which forms a datum or fixture relative to which movement of the probe assembly 23 is measured. On the platform is placed a drive system 30 having three motors, on for movement in the X-direction 140, one for the Y-direction 135 and one for the Z-direction 130. Whilst a number of different actuators could be used, in this case, these motors use ball screws to effect the three linear movements. Three sets of linear guide systems are placed orthogonally to guide three linear motions along X, Y, Z axes.

There are two revolute joints, 115 and 120, which are passive joints and together form a rotational engagement between the mounting and the probe assembly 23. Said joints permit rotation of the probe assembly 23 in the XY and YZ planes. The probe assembly is further attached to the mounting through a linkage, with the linkage coupling 100 the probe assembly at a point intermediate the extreme ends of the assembly 23. The linkage is engaged at an opposed end to the platform of the mounting. Accordingly, the probe assembly 23 is attached to the mounting at two points 25, 100, with one engagement subject to the forces and movement of the drive system 30, and the other 100 fixed to the platform, and so fixed from translational movement relative to the platform. In this arrangement, the probe assembly 23 is capable of movement in 5 degrees of freedom (X, Y, Z, XY & YZ) at one point 25 and capable of 3 degrees of freedom (XY, YZ, XZ), and so all rotational, at the other 100. A probe datum 110 is placed at the proximal end of the probe to make sure the probe assembly 23 is attached in only one position

For clarity, the first point will be referred to as the rotational engagement 25 and the second the pivotal coupling 100.

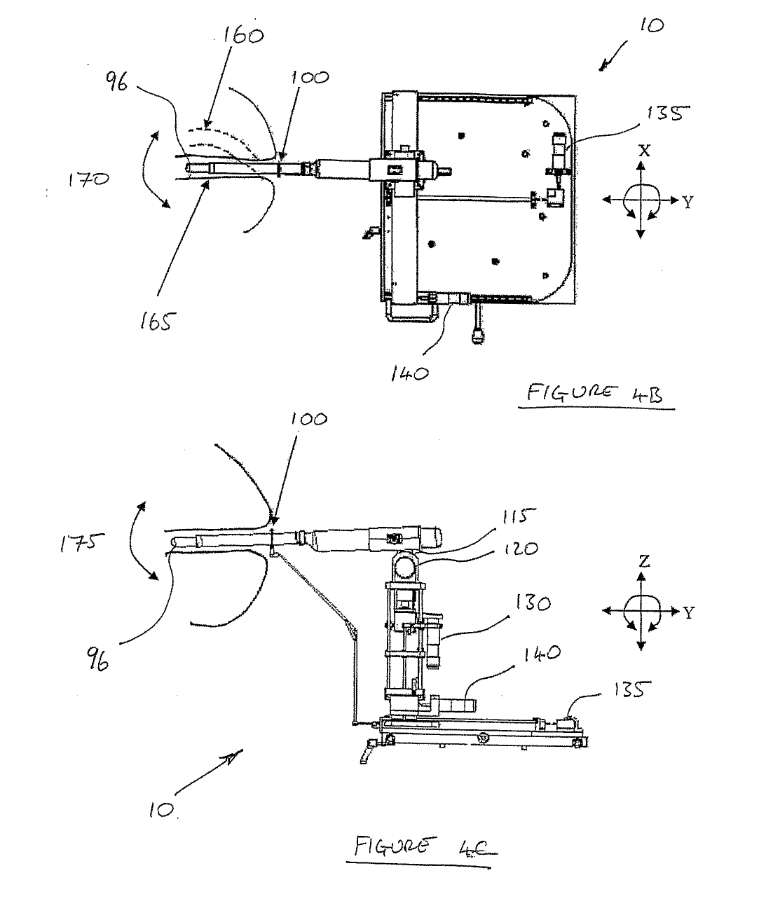

When the Z and Y axes 135, 130 move, the two passive joints 115 and 120 rotate respectively, using the pivotal coupling 100. The coupling is then placed close to patient's anus as fulcrum, to adjust the transducer to the rectal centroid of within the 6 cm interval as shown in FIGS. 4A, B, and C.

It follows, from this arrangement, that by operating the motors 140, 135 & 130 separately or in combination, the probe assembly 23 is capable of a wide range of movement.

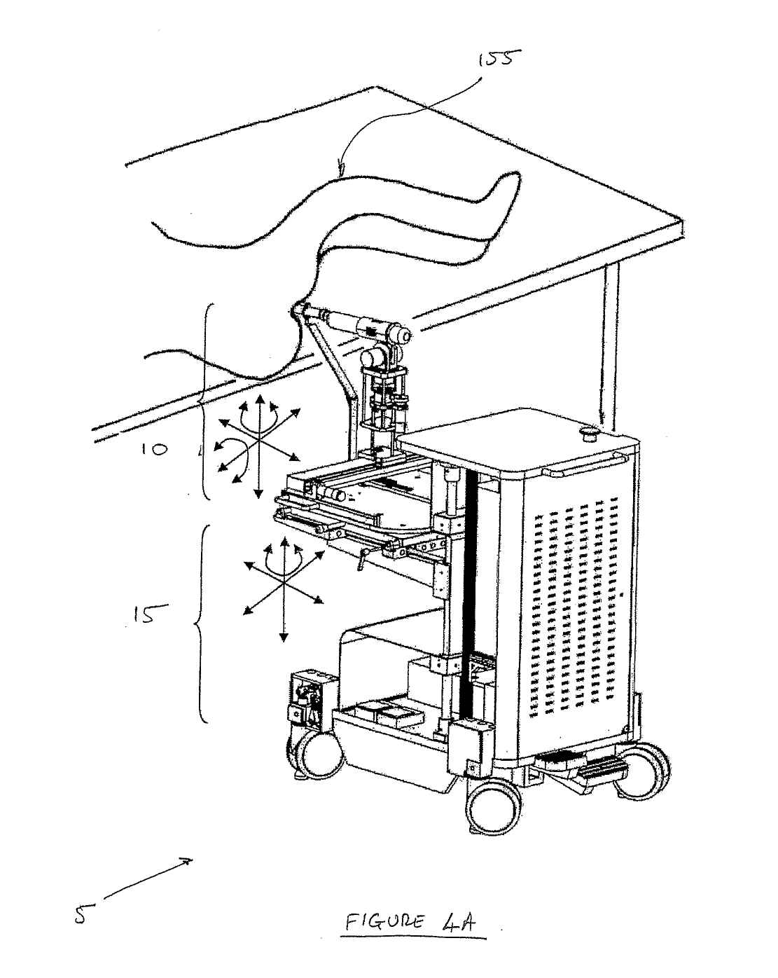

As shown in FIGS. 2, 3A and 3B, the probe assembly 23 comprises an anorectal probe 20, having a gripping end at which the mounting grips the probe at the rotational engagement 25 and an insertion end 96 which is the leading point for insertion of the probe assembly 23. Located at or near the insertion end is an ultrasound transducer for acquiring ultrasound images from tissue within a known distance from the transducer. The type and operation of the probe 20 does not, of itself, form a part of the invention, and such probes are well known and widely used in the industry for just such examinations.

The probe assembly further includes an inflatable membrane, similar to a condom which fits over the insertion end, and extends down the length of the probe 20. The inflatable membrane 250, more clearly seen in FIG. 9, is inflated with water or other liquid when the probe as been inserted. It serves the purpose of expanding the rectal void so as to more clearly examine the rectum of the patient.

A sigmoidoscope 95 is also present into which the probe 20, with the inflatable membrane, slides into position. The assembly in this arrangement is common and forms part of the art. However, as shown in FIGS. 3A and 3B, further included is a further embodiment of the present invention, being an adaptor 105. The adaptor serves the purpose of engaging the sigmoidoscope 95 and probe 20 so that they work as a single object. The benefits provided by this arrangement will be discussed in further detail later.

An objective of the invention is to mimic a skilled surgeon in performing anorectal ultrasound scanning. To achieve this objective, the invention, as shown in FIG. 1 is sufficiently stable and stiff as well as providing an ergonomically friendly set up to be handled by the surgeon.

The system 10 may be made mobile by means of a trolley 15, which includes a cart 70 so that it could be pushed by the handle 80 and stationed at desired position and orientation as shown in FIGS. 4A, 4B and 4C. For added stability, the four-wheel cart 70 has rubber padded retractable legs 65 that enable the system rest on the flour on the rubber pads, the wheels to be lifted up slightly by pushing down the paddle lock 90 when the system is pushed to desired position and orientation.

To facilitate the ease of operating the probe assembly 23, a four degree of freedom passive platform 35 is used to fine tune and lock the drive system 30 at desired position and orientation as shown in FIG. 1. A lead screw 50 driven by a bi-direction electrical motor 55 with power off breaker is used to adjust the vertical position of the drive system 30. The direction of motor is controlled by two foot switches 60. Just below the platform 35 are two pairs of orthogonally placed linear guide poles and linear bearings which allow the platform to move freely in the X-Y plane. Two lever locking devices 45 are used to freeze and unfreeze the movements. Between the platform and drive system 30 is a turning table which allows the drive system 30 to rotate freely 170 about Z axis. A knob locking device is use to lock and unlock the rotation.

The X and Y linear movements 140, 135 are used to retract the probe back segment by segment tracing an “S” shape 160 according to the natural of human rectum anatomy 165. This motion is controlled by an embedded CPU 85 and an emergency stop switch 75 will be activated by a surgeon according to his/her judgment.

While the probe is retracted, the pivot ring will move along the probe. The force acting on the anus is minimized, and so assisting in making the patient feel more comfortable.

In order to measure the distance between the pivot ring 100 and drive system 30 for control purpose, a vernier 145 is built between two devices and a knob-locking device 150 is used to lock the vernier at desired position.

According to the preferred embodiment, the methodology of the present invention is represented as follows:

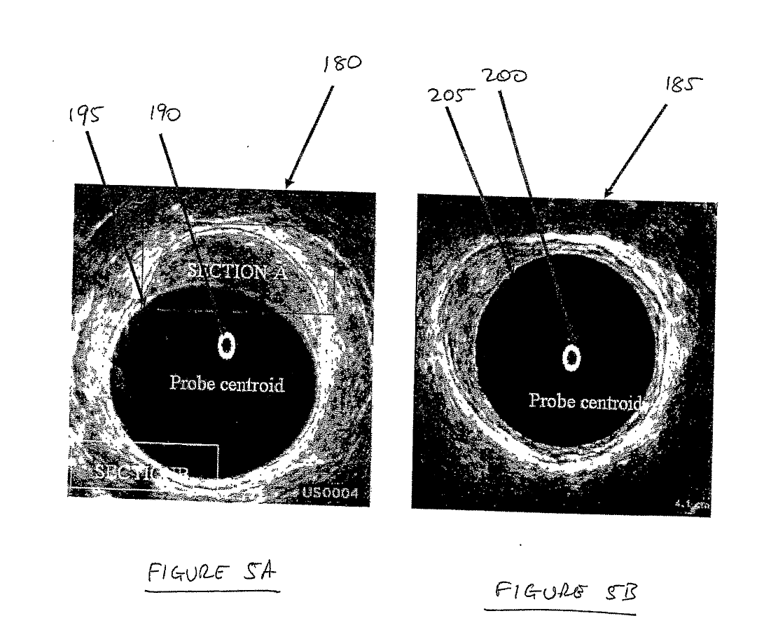

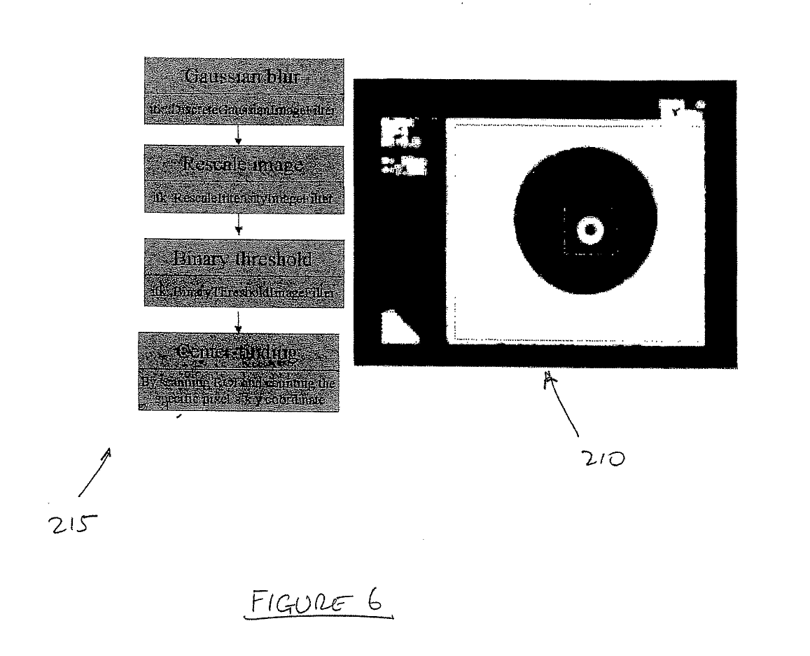

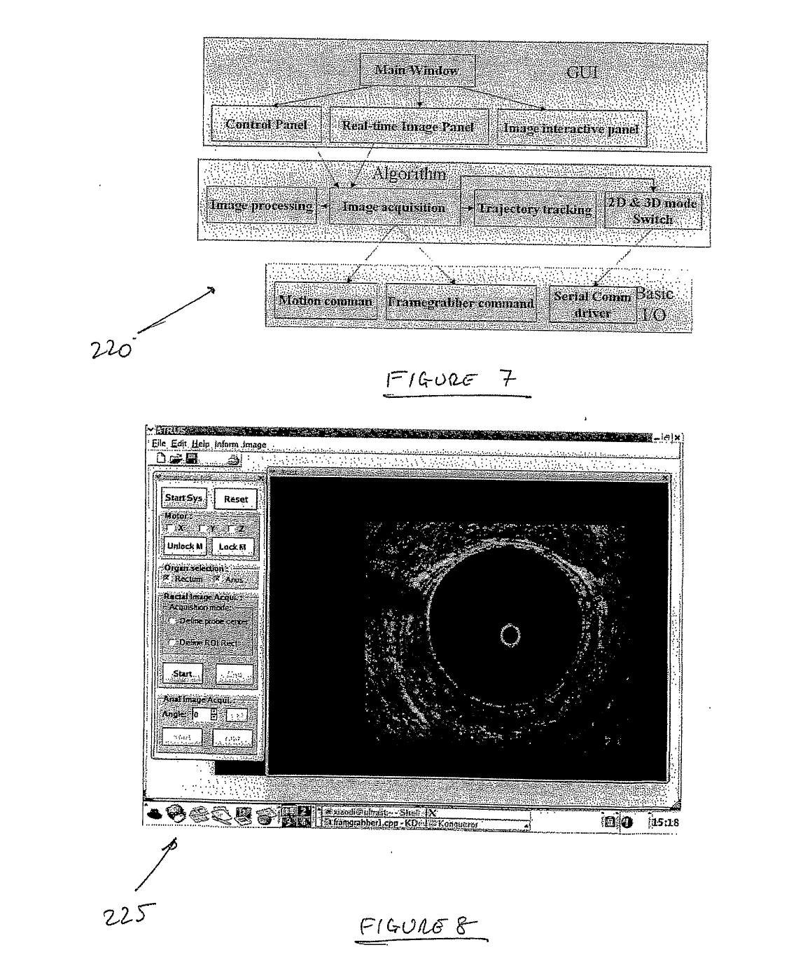

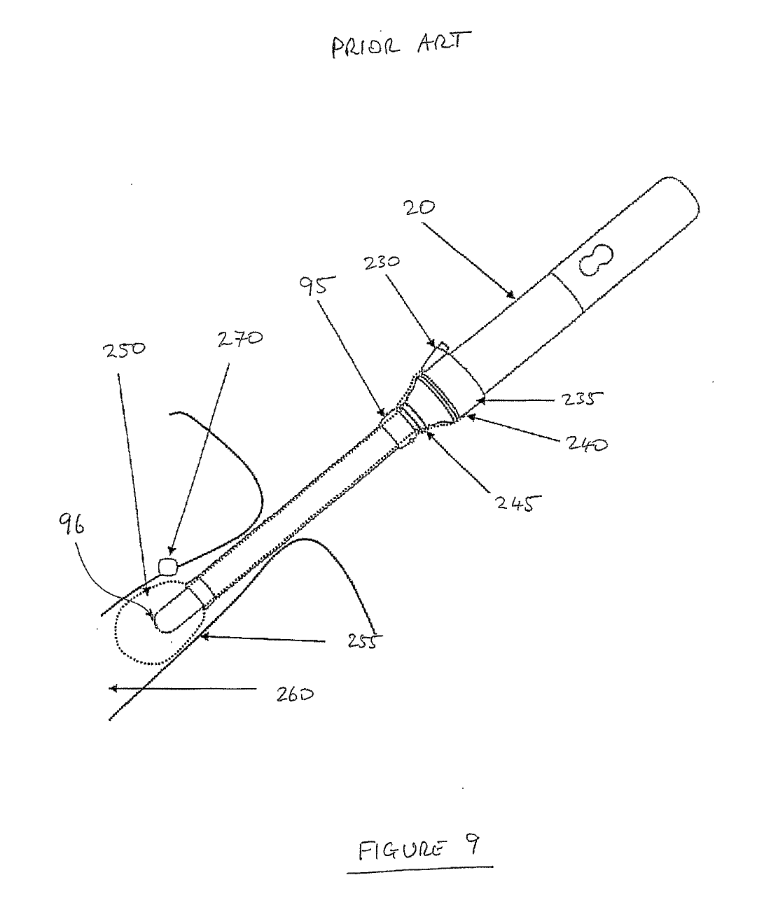

A. Positioning of anorectal examination system with respect to patient (FIG. 4A)
i. The anorectal examination system is reset to home position;
ii. Patient is made to lie on the left-lateral position such that anus area is near to the edge of the operating table;
iii. The anorectal examination system is pushed to where the probe is at the same level with the patient's anus;
iv. The cart wheels are locked by depressing the foot pedal;
v. The pivot ring is pushed close to the patient's anus;
vi. The vernier is locked and the reading will be recorded.
   B. Attaching the probe to the anorectal examination system
i. The sigmoidoscope is inserted into the rectum through the pivot ring and patient's anus;
ii. The probe, covered by a condom, is inserted into rectum through the sigmoidoscope;
iii. The sigmoidoscope and the probe are connected together by means of an adaptor as shown in FIGS. 3A, B;
iv. The height of the platform is adjusted to position the probe nearer to probe holder. Finer adjustments of the platform in X, Y axes and rotation about Z axis are performed by the respective mechanisms 40, 45 so as to fit the probe into probe holder;
v. The probe is secured onto the probe holder.
   C. Acquisition of ultrasound images (FIGS. 5A, 5B, 6, 7 and 8)
i. The anorectal examination system is activated to begin capturing ultrasound image of rectum 180, 185;
ii. The anorectal examination system computer finds out the centroid of rectum 195, 205 and adjusts the probe to this position 190, 195
iii. The anorectal examination system computer captures 210, 225 and records down this desired image. The ultrasound transducer is withdrawn to next interval;
iv. Repeating steps 2 to 4 until a 6 cm segment is completed
v. The drive system 30 pulls back the probe to the next 6 cm following “S” shape 160 trajectory as shown in FIG. 4B
vi. Repeating steps 2 to 6 until the whole rectum is scanned.
vii. Detaching the probe from probe holder

With reference to FIG. 9, a conventional ultrasound probe assembly comprises an ultrasound probe 20, water stand 235 and a sigmoidoscope 95. The water stands 235 connects to the ultrasound probe 20 mechanically by means of “O-Rings” which seal against water pressure during inflation of the membrane 250. During the scanning, it is necessary to hold the probe 20, water stand 235 and sigmoidoscope 95 together.

FIG. 10 shows an alternative arrangement, according to one embodiment of the present invention. In addition to the components comprising a conventional probe assembly, an adaptor 275 is included to obviate the need for an assistant to provide an extra set of hands to the surgeon conducting the examination. Whereas previous, the assistant is required to hold the assembly together, now the use of the adaptor 275 facilitates the holding together of the assembly. Accordingly, the three parts ultrasound probe 20, water stand 235 and sigmoidoscope 95 are connected together mechanically. Hence, the three parts will move together when the surgeon withdraws the probe 20 during anorectal ultrasound scanning.

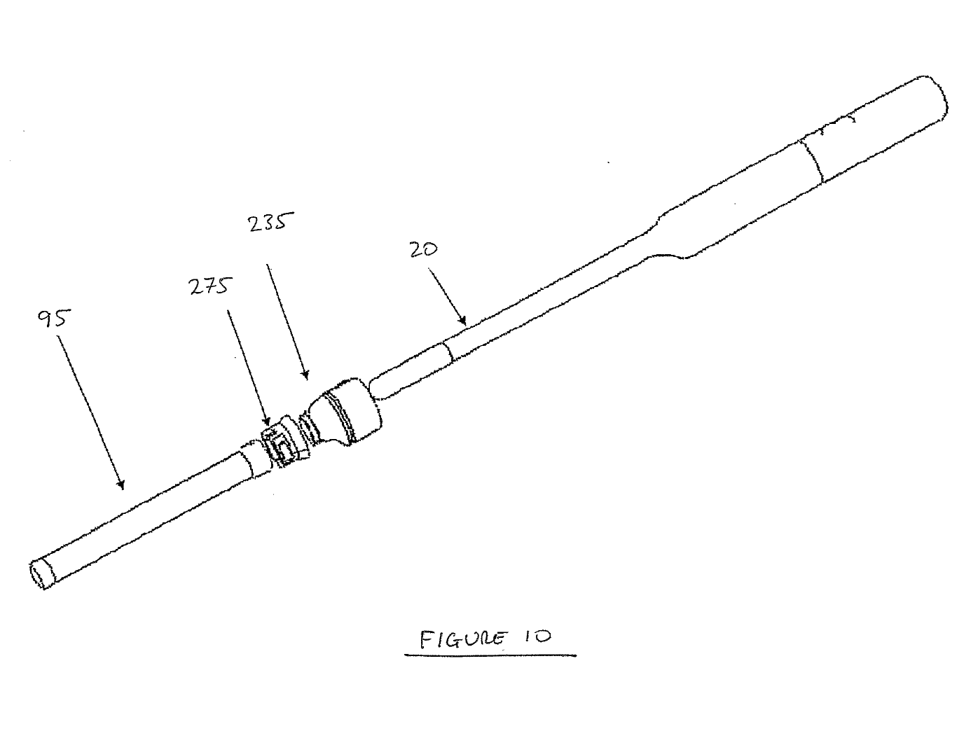

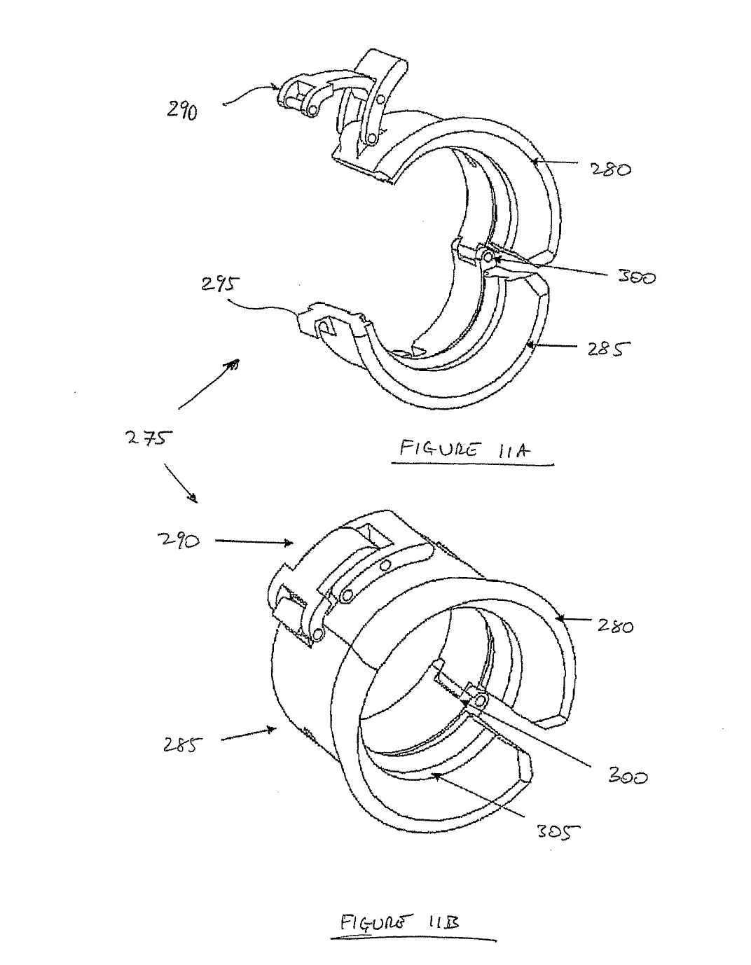

FIG. 11A shows the unlocked position of the adaptor 275. The locking latch 290 is in an unlocked position. The top half 280 and the bottom half 285 can rotate about the hinge 300. An alternative arrangement may include two halves which may be press fit together.

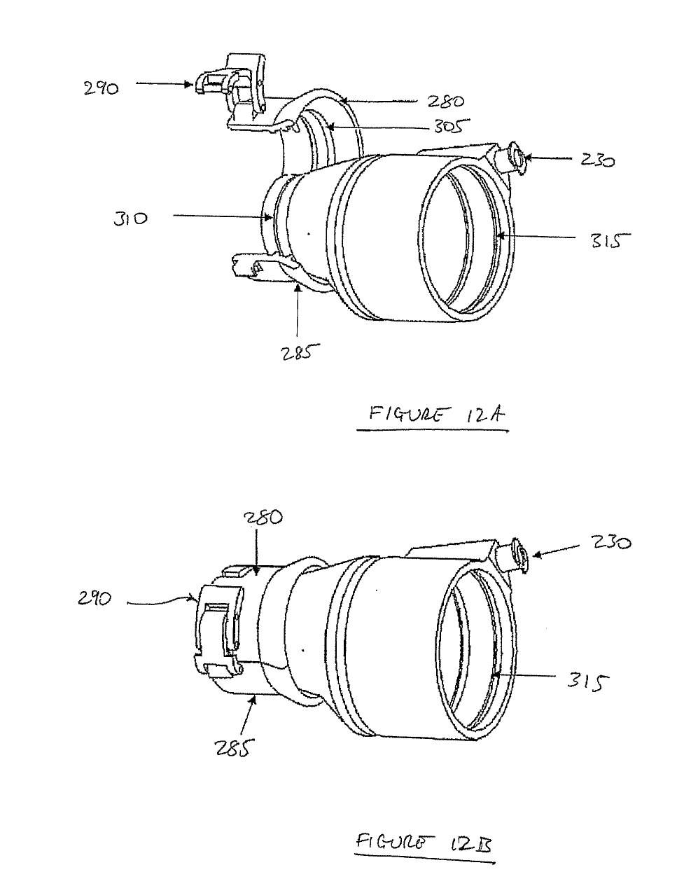

11B shows the locked position of the adaptor 275. The top half 280 and the bottom half 285 are fully engaged, with the locking mechanism 290 engaged with the corresponding latch 295. In the closed arrangement, an inner profile 305 is formed, which is shaped to engage with a corresponding external profile 310 of a portion of water stand 235 to fix the adaptor 275 with the water stand 235 as shown in FIGS. 12A and 12B. The external profile 310 is used to secure the inflatable membrane 250 at the water stand 235. The locking device 290 is used to fasten the top half 15 and the bottom half 16.

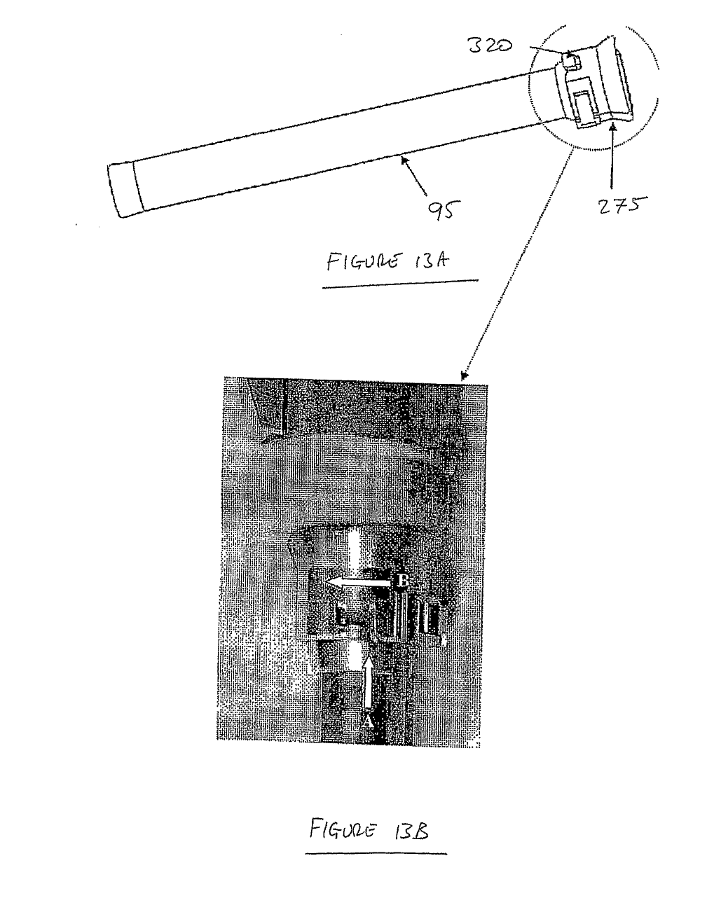

Referring to FIG. 13A, the adaptor 275 includes two slots 320 which facilitate securing and unsecuring to the sigmoidoscope 95 onto the adaptor 275. For installation, the sigmoidoscope 95 is inserted along arrow A and turn along arrow B as shown in FIG. 13B. This arrangement may be generally referred to as a bayonet fitting. Alternative arrangements may include a screw threaded engagement, a press fit or interference fit, which relies on frictional engagement.

The adaptor 275 may be made of durable and light materials with enough stiffness and corrosion resistance which include, but not limited to, surface hardened aluminum alloys or Delrin.

One such EUS procedure to operate the probe assembly using the adaptor 275 according to an embodiment of the present invention is as follows:
i. The water stand 235 is fixed on the probe 20 by means of an “O-Ring” arrangement;
ii. The probe 20 is covered with an inflatable membrane 250 that is secured at the water stand 235 with two rubber rings 245.
iii. About 100 cc of water is next used to fill up the membrane 250 and any air bubbles in the system are aspired through the water inlet 230.
iv. The adaptor 275 is installed with the water stand 235.
v. The sigmoidoscope 95 is inserted into patient's rectum 255 and the probe 20 is introduced through the sigmoidoscope 95.
vi. The sigmoidoscope 95 is slotted into the adaptor 275.
vii. More water is introduced into the membrane 250 in order to ensure that there is optimum contact between the rectal walls and the membrane, creating and enlarged rectal cavity 260;
viii. The surgeon adjusts the probe 20 by one hand to achieve a desirable quality of image captured while the ultrasound probe 20 is gradually withdrawn.

1. An anorectal probe system comprising:
an anorectal probe assembly having an insertion end for insertion into a patient's rectum, said probe assembly including a transducer at or adjacent to the insertion end for collecting ultrasound data;
a mounting in rotational engagement with said probe assembly at a point distal from the insertion end;
said mounting pivotally coupled to the probe assembly at a point intermediate the insertion end and rotational engagement point; and
wherein on application of a moment about said pivotal coupling, said mounting and probe assembly are arranged to permit selective pivotal movement of the probe assembly about the coupling.

2. The system according to claim 1 further including a drive system for applying a force along each of three principal axes to the probe assembly at the engagement point.

3. The system according to claim 2 wherein the drive system is arranged to apply forces along any two of said three principal axes and consequently apply said moment.

4. The system according to claim 2 wherein said mounting includes members movable along said principal axes and said drive system includes motors associated with said members such that upon activation of said motors, said members are advised to move along said axes and thus apply associated forces to said probe assembly.

5. The system according to claim 1 wherein said mounting includes a platform fixed relative to said members such that the mounting is pivotally coupled to the probe assembly through an assembly connecting said platform to said probe.

6. The system according to claim 1 wherein said system is mountable to a trolley for delivering said system to a desired location.

7. The system according to claim 2, wherein said drive system is arranged to apply a withdrawal force to said probe so as to incrementally withdraw said probe assembly from the patient's rectum.

8. The system according to claim 2 further including a control system for controlling the drive system such that movement and rotation of said probe assembly is subject to operation of the drive system by said control system.

9. The system according to claim 8 wherein said control system is arranged to withdraw the probe assembly such that the insertion end follows a predefined path.

10. An anorectal probe assembly comprising:
an anorectal probe;
an inflatable membrane positioned over said probe;
a water insertion system for inflating the membrane with water; and
a sigmoidoscope having a central bore for receiving the probe and inflatable membrane and an adaptor selectively engageable with said sigmoidoscope and said probe so as to fix said sigmoidoscope and probe and seal said membrane.

11. The assembly according to claim 10 wherein said adaptor is integral with said probe.

12. The assembly according to claim 10 wherein said adaptor is integral with said sigmoidoscope.

13. The assembly according to claim 10 wherein said probe and/or sigmoidoscope are selectively engageable with said adaptor through any one or a combination of: screw thread, press fit, interference fit and bayonet fitting.

14. The assembly according to claim 10 wherein said adaptor comprises two portions in hinged engagement and a connector such that engagement with one of said probe and sigmoidoscope includes hingedly clamping one of said probe and sigmoidoscope and fixing said adaptor in place with the connector.

15. A method for centralizing an anorectal probe within a patient's rectum, said probe having an insertion end with an ultrasound transducer at or adjacent to the insertion end for collecting ultrasound data, the method comprising the steps of:
positioning the insertion end within the patient's rectum;
acquiring an ultrasound image from said transducer locating a centroid of said transducer from the image;
locating a centroid of the rectal void from said image; and
calculating the difference in position of said centroids and instructing a controller to move said probe such that the centroids coincide.

16. The method according to claim 15, further including the step of inflating an inflatable membrane within the rectum so as to increase the size of a rectal void in which the transducer is positioned.

17. A method of creating a 3D ultrasound image of a patient's rectum including the steps of:
inserting an anorectal probe assembly in a patient's rectum;
controlling movement of said probe assembly using a control system in communication with a drive system;
positioning said probe such that a centroid of a transducer within said probe coincides with a centroid of a rectal void;
acquiring a two dimensional ultrasound image at a first position within said patient's rectum; and
controlling said probe so as to withdraw the transducer to a next position, repeating said positioning, acquiring and withdrawing steps a predetermined number of iterations and combining said 2 dimensional images so as to create a 3 dimensional image.
